(19) United States
(12) Patent Application Publication
(10) Pub. No.: US 2026/0211901 A1
(43) Pub. Date: Jul. 23, 2026

(54) METHOD AND ANALYZER FOR DENSITY-BASED COAL QUALITY PARAMETER ANALYSIS USING BIG DATA

(71) Applicants: Sen Di Humbert, San Diego, CA (US); Shengcai Di, Beijing (CN)

(72) Inventors: Sen Di Humbert, San Diego, CA (US); Shengcai Di, Beijing (CN)

(21) Appl. No.: 19/569,664

(22) Filed: Mar. 17, 2026

Related U.S. Application Data
(63) Continuation of application No. PCT/CN2024/091192, filed on May 6, 2024.

(30) Foreign Application Priority Data
Oct. 23, 2023 (CN) 202311421708.X

(51) Int. Cl. G06F 16/28; G01N 33/22

(52) U.S. Cl. CPC G06F 16/284; G01N 33/222

(57) ABSTRACT

The present invention discloses a density-based coal quality parameter analysis method, analyzer, and system using big data. The method is based on the theoretical equation

$${ρ}_{coal} ( \frac{1 g}{{v}_{g}} ) = {ρ}_{A} * A + {ρ}_{M} * M + {ρ}_{V} * V + {ρ}_{F C} * FC ,$$

collect a large number of data on ash (A), moisture (M), volatile matter (V), and fixed carbon (FC), calorific value (Q) and sulfur(S), construct coal quality parameter databases, develop the big data-driven analytical software application. The software receives the coal parameter variation signal U measured by the coal parameter measurement devices, identifies the corresponding coal quality parameters A, M, V, FC, Q, and S. The present Invention does not require the construction of a measurement mathematical model and is unaffected by variations in coal type or coal quality, ensuring high measurement accuracy. It is suitable for both online and offline measurement of coal quality parameters.

This application claims the benefit of priority to international application No. PCT/CN2024/091192, filed on 6 May 2024, and published as WO2025086595A1, on 1 May 2025, which claims the benefit of priority to Chinese Patent Application No. CN202311421708.X, filed on Oct. 23, 2023, titled “Method and Analyzer for Density-Based Coal Quality Parameter Analysis,” each of which is incorporated by reference herein in its entirety.

### TECHNICAL FIELD

The present invention relates to the measurement of coal quality parameters, including ash (A), moisture (M), volatile matter (V), fixed carbon (FC), calorific value (Q), and sulfur(S). Specifically, it introduces the method and analyzers for analyzing coal quality parameters by measuring the density

$${ρ}_{coal} ( \frac{1 g}{{v}_{g}} )$$

of coal or coal samples and applying big data analysis techniques to extract and interpret the coal quality parameters.

### BACKGROUND

Currently, there are several methods for analyzing and measuring coal quality parameters:

### Elemental Analysis Method

This approach characterizes coal as a composite of elemental constituents—primarily carbon (C), hydrogen (H), oxygen (O), nitrogen (N), and sulfur(S). Common detection techniques include neutron-induced prompt γ-ray analysis, neutron activation analysis, and X-ray fluorescence spectroscopy, each requiring specialized instrumentation. However, due to the complexity, high cost, and radiation safety concerns associated with these systems, their practical applications remain limited.

### Proximate Analysis of Coal

This method characterizes coal in terms of four primary components: ash (A), moisture (M), volatile matter (V), and fixed carbon (FC). The prevailing industry standard for this analysis is GB/T 212-2008, which utilizes an ashing technique to quantify these parameters. While this approach is widely adopted and remains the most used, its principal limitation lies in the lengthy testing duration, making it less suitable for real-time monitoring and process control in production environments.

### X-Ray Method for Online Measurement of Ash, Moisture, and Calorific Value

This technique employs X-ray detection to determine ash content and microwave sensing to measure moisture. Calorific value is then calculated based on the measured parameters. As the underlying technology and associated instrumentation are relatively recent developments, their adoption across the industry remains limited.

### Dual-Energy Gamma-Ray Ash Online Measurement Method

This technique determines ash content by measuring the differential absorption of γ-ray energy by ash constituents. It is widely adopted in international applications for real-time monitoring. However, despite its broad usage, the method exhibits significant limitations, most notably, concerns related to radiation safety and reduced adaptability to varying coal types and compositions.

### Patent CN116773398A

This publication describes a coal quality parameter measurement method based on coal density; wherein mathematical models are established to correlate ash (A), moisture (M), volatile matter (V), and fixed carbon (FC) with coal density as the independent variable. These models enable indirect estimation of the specified parameters. However, as the patent remains in the application phase, the disclosed method has not yet been implemented in practical industrial settings.

In addition, other methods such as microwave, laser, neutron activation, and X-ray fluorescence exist but are seldom used.

This patent application discloses a density-based method for analyzing coal quality parameters, enhanced by big data analytics to establish comprehensive coal quality databases and develop intelligent analysis software application. The software interprets variation signals (U) generated by coal quality parameter measurement devices, enabling accurate identification and assessment of key coal quality parameters.

### BRIEF DESCRIPTION OF THE SEVERAL VIEWS OF THE DRAWINGS

FIG. 1 illustrates a schematic diagram of a coal quality parameter analyzer constructed in accordance with embodiments of the present invention.

FIG. 2A illustrates a schematic diagram of various types of coal quality parameter analyzers constructed in accordance with the methods described in the embodiments of the present invention.

FIG. 2B illustrates a schematic diagram of various specific coal quality parameter analyzers constructed using different density measurement devices, in accordance with embodiments of the present invention.

FIG. 3 illustrates a schematic diagram of an offline density coal quality parameter analyzer, as described in embodiments of the present invention.

FIG. 4 illustrates a schematic diagram of an online electronic belt scale coal quality parameter analyzer, as described in embodiments of the present invention.

FIG. 5 illustrates a schematic diagram of an X-ray scale coal quality parameter analyzer, as described in embodiments of the present invention.

FIG. 6 illustrates a schematic diagram of an online nuclear scale coal quality parameter analyzer, as described in embodiments of the present invention.

FIG. 7 illustrates a schematic diagram of an online microwave coal quality parameter analyzer, as described in embodiments of the present invention.

FIG. 8 illustrates a schematic diagram of an online laser scale coal quality parameter analyzer, as described in an embodiment of the present invention.

### DETAILED DESCRIPTION

### Summary

To address the limitations of conventional coal quality measurement methods, specifically low accuracy and extended analysis times, the present invention provides:
1. A big data-enabled density-based method for analyzing coal quality parameters, and
2. an analyzer constructed in accordance with the above method, including the integrated coal quality parameters system for real-time measurement, and data storage medium to support the acquisition, processing, and interpretation of measurement data.

### Scientific Foundation

Proximate Analysis of Coal (GB/T 212-2008): According to the GB/T 212-2008 standard, coal is defined as comprising four primary constituents: ash (A), moisture (M), volatile matter (V), and fixed carbon (FC). Each parameter—A, M, V, FC—represents the mass percentage of its respective component (W_{A}, W_{M}, W_{V}, W_{FC}) relative to the total mass W_{coal} of the coal sample, expressed as:

$$A = \frac{{w}_{A}}{{w}_{coal}} * 100 \% , M = \frac{{w}_{M}}{{w}_{coal}} * 100 \% , \\ V = \frac{{w}_{v}}{{w}_{coal}} * 100 \% , FC = \frac{{w}_{FC}}{{w}_{coal}} * 100 \% \\ {W}_{coal} = {W}_{A} + {W}_{M} + {W}_{V} + {W}_{FC}$$

Furthermore, A+M+V+FC=100%

Under the GB/T 212-2008 standard, the ashing technique is employed to determine the coal quality parameters—ash (A), moisture (M), volatile matter (V), and fixed carbon (FC). A 1-gram coal sample is weighed, combusted in a muffle furnace, and dried in a ventilated oven to determine the mass of each component: W_{A}, W_{M}, W_{V}, W_{FC}. These measured values enable the calculation of the corresponding percentage parameters relative to the total sample weight.

$$A = \frac{{w}_{A}}{{1}_{g}} * 1 \; 00 \% M = \frac{{w}_{M}}{{1}_{g}} * 1 \; 00 \% V = \frac{{w}_{v}}{{1}_{g}} * 1 \; 00 \% FC = \frac{{w}_{F C}}{{1}_{g}} * 100 \%$$

For coal samples of equal mass (1 gram), variations in the measured values of ash (A), moisture (M), volatile matter (V), and fixed carbon (FC) are consistently observed. This indicates that mass alone is insufficient to characterize these parameters. The volume Vg occupied by the 1-gram sample must also be considered. Differences in this volume—expressed as

$$\frac{{1}_{g}}{{v}_{g}} ,$$

—result in variations in the sample's density

$${ρ}_{coal} ( \frac{1 g}{{v}_{g}} ) ,$$

which in turn influence the measured coal quality parameters A, M, V, and FC.

Accordingly, the present invention introduces a new understanding: coal is characterized not solely by the individual masses of its constituent components, but also by the volumes those masses occupy. In essence, coal is defined by the densities of its components ρ_{A}, ρ_{M}, ρ_{V}, ρ_{FC}. The overall density of the coal sample is thus determined by the weighted contribution of each component's density, modulated by its respective mass fraction (A, M, V, FC).

$${ρ}_{coal} ( \frac{1 g}{{v}_{g}} ) = {ρ}_{A} * A + {ρ}_{M} * M + {ρ}_{V} * V + {ρ}_{FC} * FC \qquad ( 2 )$$

where:

$${ρ}_{coal} ( \frac{lg}{Vg} )$$

denotes coal or coal sample's density;
A, M, V, and FC denote coal quality parameters, including ash, moisture, volatile matter, fixed carbon, respectively;
Q denotes calorific value of coal; S denotes sulfur content in coal; and
ρ_{A}, ρ_{M}, ρ_{V}, and ρ_{FC} denote ash density, moisture density, volatile matter density, and fixed carbon density of coal, respectively.

In equation (2), ρ_{A}>ρ_{M}>ρ_{V}>ρ_{FC} are assumed as constants;

Therefore,

$${ρ}_{coal} ( \frac{1 g}{{v}_{g}} ) = f ( A , M , V , FC ) ,$$

that is,

$${ρ}_{coal} ( \frac{1 g}{{v}_{g}} )$$

is a function of the percentage variations in A, M, V, and FC. This formulation implies that variations in

$${ρ}_{coal} ( \frac{1 g}{{v}_{g}} )$$

directly reflect changes in the parameters (A, M, V, FC)ᵢ.

This functional relationship forms the theoretical basis of the present invention's density-based coal quality parameter analysis method. Similarly, studies of the calorific value determination method (GB/T 213-2008) and the total sulfur determination method (GB/T 214-2007) reveal that both use 1-gram coal sample (more precisely, represented as

$$( \frac{1 g}{{v}_{g}} )$$

to measure calorific value (Q) and sulfur content (S). Therefore, variations in

$$( \frac{1 g}{{v}_{g}} )$$

can likewise represent changes in Q and S. Hence, changes in

$$( \frac{1 g}{{v}_{g}} )$$

serve as a unified indicator for characterizing variations across the full set of coal quality parameters (A, M, V, FC, Q, S)ᵢ.

### Technical Concept

Based on the theoretical equation

$${ρ}_{coal} ( \frac{1 g}{{v}_{g}} ) = {ρ}_{A} * A + {ρ}_{M} * M + {ρ}_{V} * V + {ρ}_{FC} * FC ,$$

a quantitative relationship is established between coal density

$${ρ}_{coal} ( \frac{1 g}{{v}_{g}} )$$

and coal quality parameters (A, M, V, FC, Q, S)ᵢ. Using the big data analytics technologies to build corresponding databases to map

$${ρ}_{coal} ( \frac{1 g}{{v}_{g}} )$$

with (A, M, V, FC, Q, S)ᵢ.

To determine coal density

$${ρ}_{coal} ( \frac{1 g}{{v}_{g}} ) ,$$

analyzers based on existing measurement technologies—such as X-ray, γ-ray can be utilized. These devices output a variation signal U, which inherently reflects changes in the coal sample's density

$${ρ}_{coal} ( \frac{1 g}{{v}_{g}} ) .$$

By constructing a database that correlates the signal U with corresponding values

$${ρ}_{coal} ( \frac{1 g}{{v}_{g}} ) ,$$

the coal density can be accurately determined from the measured signal U.

Using recognition technology, the present invention introduces an intelligent analysis software application capable of identifying the signal U and determining the corresponding coal density

$${ρ}_{coal} ( \frac{1 g}{{v}_{g}} ) .$$

The software then queries a pre-established database mapping

$${ρ}_{coal} ( \frac{1 g}{{v}_{g}} )$$

in the

$${ρ}_{coal} ( \frac{1 g}{{v}_{g}} ) ∼ {( A , M , V , FC , Q , S )}_{i}$$

database, thereby retrieving the associated coal quality parameters A, M, V, FC, Q, S. This approach forms the basis of the data-enabled density-based method for coal quality parameter analysis.

### Key Features of the Big Data-Enabled Density-Based Coal Quality Parameter Analysis Method

Using a coal quality parameter measurement device to measure a coal quality parameter variation signal U of coal or coal sample, the signal U serving as a variable input for analysis; and
identifying corresponding coal quality parameters, including at least one of ash (A), moisture (M), volatile matter (V), fixed carbon (FC), calorific value (Q), or sulfur (S), corresponding to a value of the signal U via a big data-driven coal quality parameter databases,
wherein the identifying coal quality parameters corresponding to U via the big data-driven coal quality parameter databases comprises:

### Step 1:

using the coal quality parameter variation signal U, measured by the coal quality parameter measurement device, as a variable; and
utilizing big data technology to construct one or more databases based on a relationship

$${ρ}_{coal} ( \frac{1 g}{{v}_{g}} ) = {ρ}_{A} * A + {ρ}_{M} * M + {ρ}_{V} * V + {ρ}_{FC} * FC ,$$

using large datasets of measured A, M, V, FC, Q, S values by applying national standards methods across countries, represented as (A, M, V, FC, Q, S)ᵢ, i=1, 2, 3 . . . n, the one or more databases being designed to store coal quality parameters and corresponding

$${ρ}_{coal} ( \frac{1 g}{{v}_{g}} )$$

and coal quality parameter variation signal U measurement data, and being adaptable to various coal types and the intelligent analysis software application analytical requirements,
where:

$${ρ}_{c o a l} ( \frac{lg}{Vg} )$$

denotes coal or coal sample's density;
A, M, V, and FC denote coal quality parameters, including ash, moisture, volatile matter, fixed carbon, respectively;
Q denotes calorific value of coal; S denotes sulfur content in coal; and
ρ_{A}, ρ_{M}, ρ_{V}, and ρ_{FC} denote ash density, moisture density, volatile matter density, and fixed carbon density of coal, respectively.

### Step 2:

employing identification technology using the variation signal U of the coal or coal sample measured by the coal quality parameter measurement devices, and statically measured coal or coal sample's quality parameters A, M, V, FC, combined with the one or more databases storing coal quality parameters, to develop an intelligent analysis software application; and

### Step 3:

constructing a coal quality parameter analyzer comprising a measurement device and an integrated control and computation unit, the coal quality parameter measurement device capturing the coal quality parameter variation signal U, transmitting the variation signal U to the control and computation unit, which processes U using the intelligent analysis software application, and through recognition and databases matching, identifies the corresponding coal quality parameters A, M, V, FC, Q, S.

### Construct Coal Quality Parameter Databases

The process of constructing one or more distinct coal quality parameter databases or parameter measurement databases using big data technologies is characterized by the following:
constructing a

$${ρ}_{coal} ( \frac{1 g}{{v}_{g}} ) ∼ {( A , M , V , FC , Q , S )}_{i}$$

database;
constructing a

$$U ∼ {ρ}_{coal} ( \frac{1 g}{{v}_{g}} )$$

database;
constructing a U~(A, M, V, FC, Q, S)ᵢ database;
constructing a

$${ρ}_{coal} ( \frac{1 g}{{v}_{g}} ) ∼ {( A , M , V , FC , Q , S )}_{i}$$

parameter measurement database;
constructing a

$$U ∼ {ρ}_{coal} ( \frac{1 g}{{v}_{g}} )$$

parameter measurement database;
constructing a U~(A, M, V, FC, Q, S)ᵢ parameter measurement database; and
constructing

$$A ∼ {ρ}_{coal} ( \frac{1 g}{{v}_{g}} ) , M ∼ {ρ}_{coal} ( \frac{1 g}{{v}_{g}} ) , V ∼ {ρ}_{coal} ( \frac{1 g}{{v}_{g}} ) , FC ∼ {ρ}_{coal} ( \frac{1 g}{{v}_{g}} ) , Q ∼ {ρ}_{coal} ( \frac{1 g}{{v}_{g}} ) , and S ∼ {ρ}_{coal} ( \frac{1 g}{{v}_{g}} )$$

parameter measurement databases.

Those skilled in the art may also construct additional coal quality parameter databases, including specialized datasets tailored to various coal types—such as bituminous coal, anthracite, lignite, imported coal, and blended coal. Furthermore, individual measurement databases may be developed for each parameter—ash (A), moisture (M), volatile matter (V), fixed carbon (FC), calorific value (Q), and sulfur (S)—based on their respective correlations with the variation signal U, as A~U, M~U, V~U, FC~U, Q~U, S~U parameter measurement databases, All such implementations fall within the scope of the present patent protection.

Coal quality parameter analyzer constructed using the big data-enabled density-based coal quality parameter analysis method (e.g., a big data-enabled coal quality parameter analyzer)

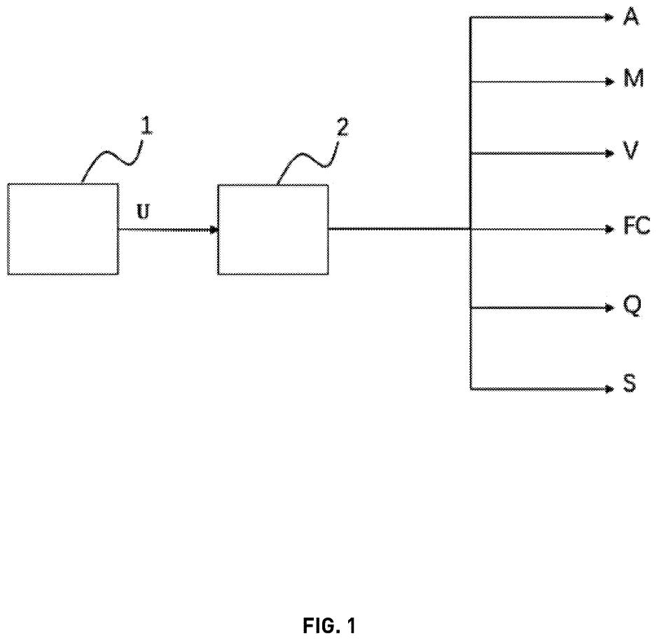

FIG. 1 illustrates the big data-enabled coal quality parameter analyzer, it comprises:
1. coal quality parameter measurement device (1) and
2. control and computation unit (2)

The type of coal quality parameter measurement device may be:
a density-based coal quality parameter measurement device (11)
an electronic belt scale-based coal quality parameter measurement device (12)
an X-ray-based coal quality parameter measurement device (13)
a γ-ray-based coal quality parameter measurement device (14)
a microwave-based coal quality parameter measurement device (15)
a laser-based coal quality parameter measurement device (16)
or any other type of coal quality parameter measurement device constructed by any suitable method, provided it is capable of measuring the coal quality parameters variation signal U.

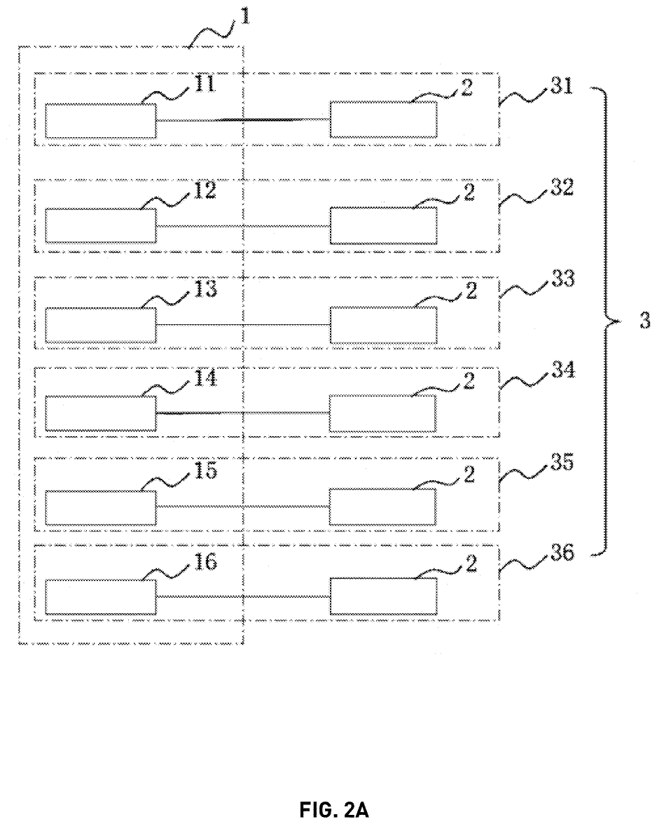

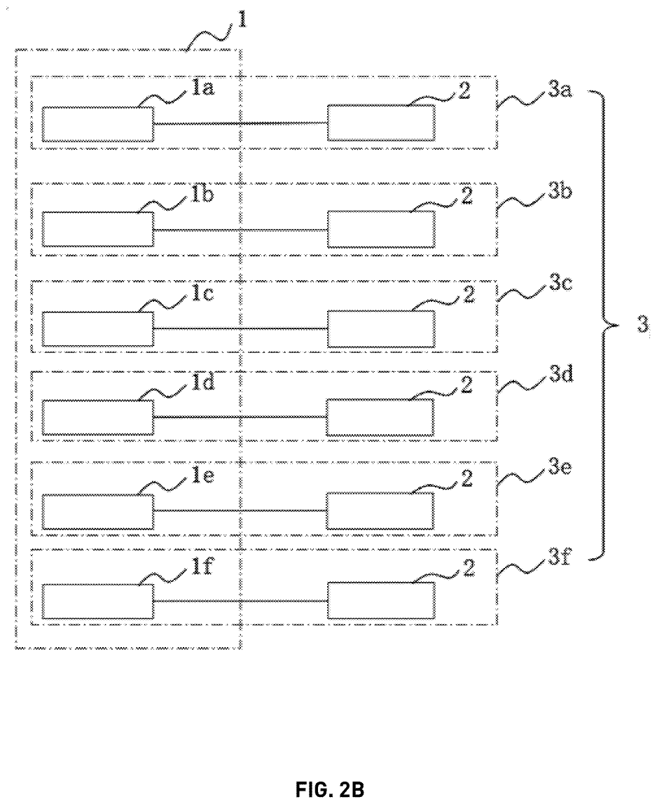

FIG. 2A illustrates various types of coal quality parameter analyzers, each formed by integrating a coal quality parameter measurement device with its corresponding method.

FIG. 2B illustrates various coal quality parameter analyzers, each assembled from specific measurement devices. For instance, the X-ray-based coal quality parameter measurement device (13) corresponds to a specific implementation—namely, the density measurement X-ray belt scale device (1c). The density measurement X-ray belt scale coal quality parameter analyzer (3c) is constructed using the density measurement X-ray belt scale device (1c) as its core measurement component. Other types of coal quality parameter analyzers are all constructed in a manner like the density measurement X-ray belt scale coal quality parameter analyzer (3c), such as:
a density measurement electronic coal quality parameter analyzer (3a)
a density measurement electronic belt scale coal quality parameter analyzer (3b)
a density measurement nuclear belt scale coal quality parameter analyzer (3d)
a density measurement microwave coal quality parameter analyzer (3e)
a density measurement laser belt scale coal quality parameter analyzer (3f)

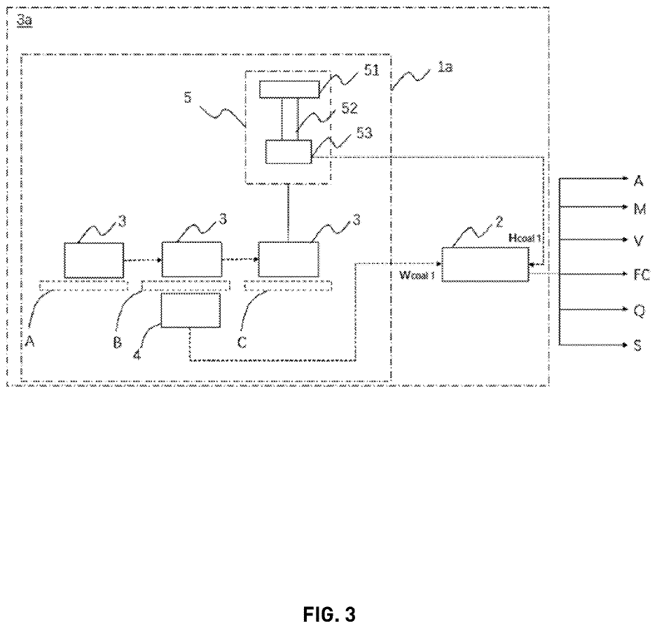

FIG. 3 illustrates a schematic diagram of an offline density measurement coal quality parameter analyzer.

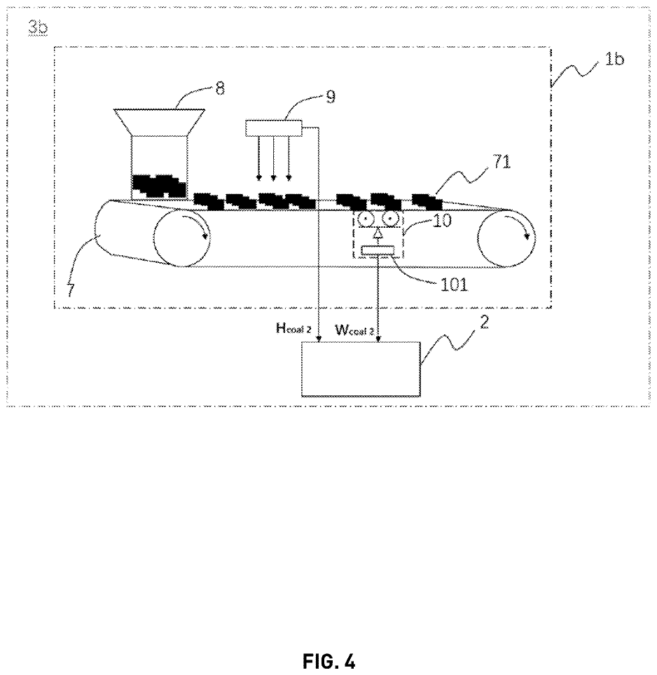

FIG. 4 illustrates a schematic diagram of an online density measurement electronic belt scale coal quality parameter analyzer.

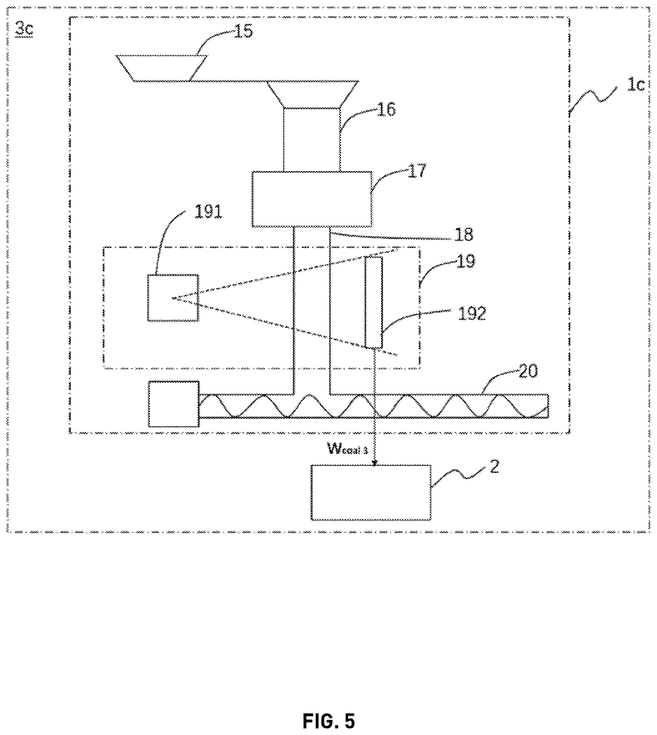

FIG. 5 illustrates a schematic diagram of an online density measurement X-ray coal quality parameter analyzer.

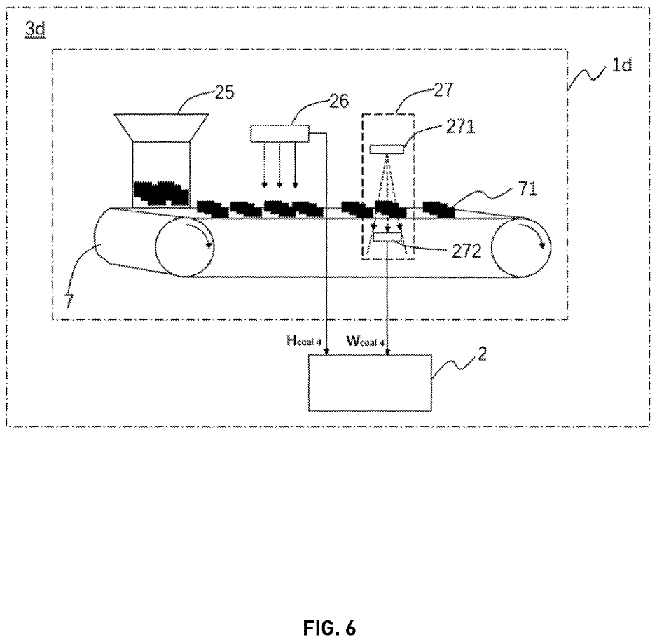

FIG. 6 illustrates a schematic diagram of an online density measurement nuclear belt scale coal quality parameter analyzer.

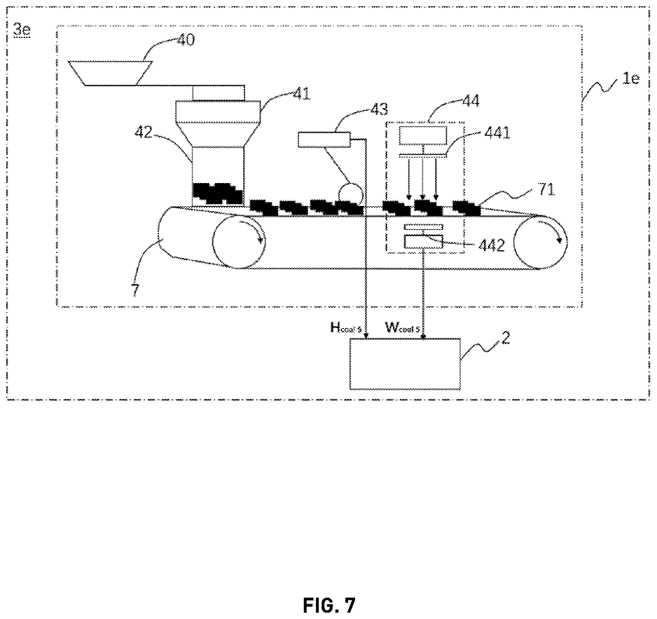

FIG. 7 illustrates a schematic diagram of an online density measurement microwave coal quality parameter analyzer.

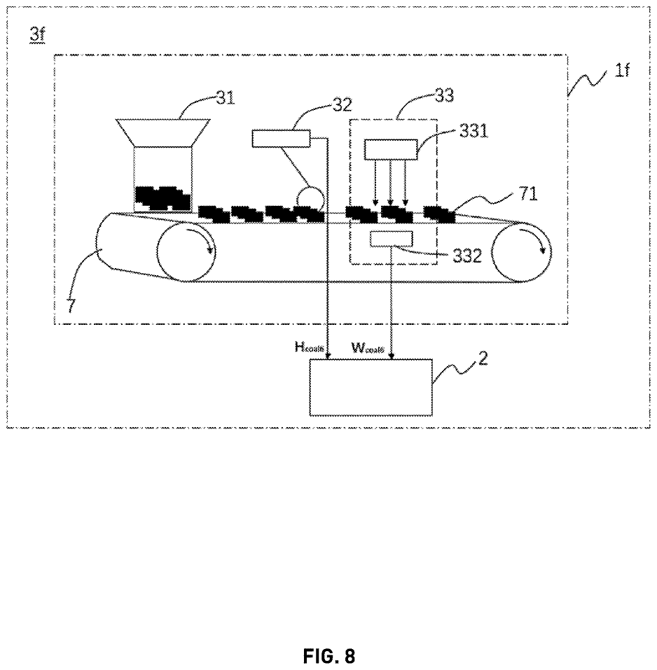

FIG. 8 illustrates a schematic diagram of an online density measurement laser coal quality parameter analyzer.

Coal quality parameter analyzer can be divided into offline and online coal quality parameter analyzer depending on the application.

The following detailed description of the invention is provided with reference to the accompanying drawings and specific embodiments, aiming to further clarify its objectives, design principles. It should be noted that the embodiments disclosed herein represent only a portion of the invention and do not constitute its full scope. Any additional embodiments that can be conceived by those skilled in the art without requiring inventive effort, are deemed to fall within the scope of this invention.

In existing technologies, any method capable of measuring the density of solid materials can be applied to determine the density of coal or coal sample.

$${ρ}_{coal} ( \frac{lg}{Vg} ) = \frac{{W}_{coal}}{{V}_{coal}} ,$$

Once W_{coal} and V_{coal} are measured, the density

$${ρ}_{coal} ( \frac{lg}{Vg} )$$

can be obtained.

### Embodiment 1—Construction of Density Measuring Device 1a

An encoder-based thickness measurement device is used to measure the height H_{coal1} of the coal sample in the weighing cup, and an electronic scale or balance is used to measure the weight W_{coal1} of the coal sample in the cup.

FIG. 3 illustrates a schematic diagram of an offline coal quality parameter analyzer constructed using the density measurement device. The density coal quality parameter analyzer 3a comprises:
a measuring cup (3) for containing the coal sample to be tested.
a weighing device for measuring the weight W_{coal1} of the coal sample.
an encoder measurement device for measuring the thickness of the coal sample H_{coal1} in the cup, comprising
motor and encoder 51
lead screw 52
pressing head 53
and a control and computation unit (2) that receives the measurement signals W_{coal1} and H_{coal1}, computes the density

$${ρ}_{coal measured 1} = \frac{{W}_{coal 1}}{{H}_{coal 1} * {S}_{1}} ,$$

where S₁ is the area of the cup bottom. (ρ_{coal measured1} corresponds to the described signal U and ρ_{coal}). Based on the ρ_{coal measured1} identifies the corresponding coal quality parameters through the analysis software application.

### Embodiment 2—Construction of an Electronic Belt Scale Density Measuring Device 1b

A multi-channel laser thickness measurement device (9) is used to measure the thickness H_{coal2} of the coal or coal sample on the conveyor belt, while an electronic belt scale is used to measure the weight W_{coal2} of the coal or coal sample on the belt.

FIG. 4 illustrates a schematic diagram of an online coal quality parameter analyzer constructed using the electronic belt scale density measurement device. The electronic belt scale analyzer 3b comprises:
a shaping hopper (8) for shaping the coal or coal sample (71) to uniform width and thickness.
a multi-channel laser thickness measurement device (9) for measuring the thickness H_{coal2} of the coal or coal sample (71) on the conveyor belt.
an electronic belt scale (10) with a pressure sensor (101) for measuring the weight W_{coal2} of the coal or coal sample on the conveyor belt.
and a control and computation unit (2) that receives the measurement signals W_{coal2} and H_{coal2}, computes the density

$${ρ}_{coal measured 2} = \frac{{W}_{coal 2}}{{H}_{coal 2} ⋆ {S}_{2}} ,$$

where S₂ is the bottom area of the measured coal sample.

(ρ_{coal measured2} corresponds to the described signal U and ρ_{coal}). Based on the ρ_{coal measured2} identifies the corresponding coal quality parameters through the analysis software application.

### Embodiment 3—Construction of an X-ray Scale Density Measuring Device 1c

An X-ray scale is used to measure the weight W_{coal3} of the coal or coal sample inside the measuring tube. Since the internal volume of the measuring tube is a constant, V_{coal3}=constant, the density

$${ρ}_{coal measured 3} = \frac{{W}_{coal 3}}{{V}_{coal 3}}$$

of the coal sample in the measuring tube can be calculated.

FIG. 5 illustrates a schematic diagram of an online coal quality parameter analyzer constructed using the X-ray scale density measurement device. The X-ray scale coal quality parameter analyzer 3c comprises:
a sampler (15) for extracting coal or coal sample from the production line.
a hopper (16) for feeding the extracted coal or coal sample into a crusher.
a crusher (17) for crushing the coal or coal sample into uniform granularity.
a measuring tube (18) for conveying the uniform coal sample.
an X-ray scale (19) including an X-ray source (191) and X-ray detector (192) for measuring the weight W_{coal3} of the coal sample inside the measuring tube (18);
a screw conveyor (20) for conveying coal sample within the measuring tube.
and a control and computation unit (2) for receiving the signal W_{coal3} and computing the density

$${ρ}_{coal measured 3} = \frac{{W}_{coal 3}}{{V}_{coal 3}} ( {V}_{coal 3} = constant ) ,$$

(ρ_{coal measured3} corresponds to the described signal U and ρ_{coal}). Based on the ρ_{coal measured3} identifies the corresponding coal quality parameters through the analysis software application.

### Embodiment 4—Construction of a Nuclear Scale Density Measuring Device 1d

A nuclear scale is used to measure the weight W_{coal4} of coal or coal sample on a conveyor belt, while a multi-channel laser thickness measurement device is used to measure the thickness H_{coal4} of the coal or coal sample on the belt.

FIG. 6 illustrates a schematic diagram of an online coal quality parameter analyzer constructed using the nuclear scale density measurement device. The nuclear scale coal quality parameter analyzer comprises:
a shaping hopper (25) for shaping the coal or coal sample (71) into a stream of uniform width and thickness.
a multi-channel laser thickness measurement device (26) for measuring the thickness H_{coal4} of the coal or coal sample on conveyor 7.
a nuclear scale (27) comprising a γ-ray source (271) and γ-ray detector (272) for measuring the weight W_{coa14} of the coal or coal sample on the belt.
and a control and computation unit (2) that receives the measurement signals W_{coal4} and H_{coal4}, computes the density

$${ρ}_{coal measured 4} = \frac{{W}_{coal 4}}{{H}_{coal 4} * {S}_{4}} ,$$

where S₄ is the bottom area of the measured coal sample.

(ρ_{coal measured4} corresponds to the described signal U and ρ_{coal}). Based on the ρ_{coal measured4}, identifies the corresponding coal quality parameters through the analysis software application.

### Embodiment 5—Construction of a Microwave Weighing Density Measuring Device 1e

A microwave measurement device is used to measure the weight W_{coal5} of coal or coal sample on the conveyor belt, and an angular-displacement thickness measurement device is used to measure the thickness H_{coal5} of the coal or coal sample.

FIG. 7 illustrates an online coal quality parameter analyzer constructed using the microwave density measurement device. The microwave coal quality parameter analyzer 3e comprises:
a sampler (40) for extracting coal or coal sample from the production line.
a crusher (41) for crushing the coal or coal sample into uniform granularity.
a shaping hopper (42) for shaping the coal or coal sample (71) into a stream of uniform width and thickness.
an angular-displacement thickness measurement device (43) for measuring the thickness H_{coal5} of the coal sample.
a microwave weighing device (44) comprising a microwave generator and transmitting antenna (441) and a receiving antenna and receiver (442) for measuring the weight W_{coal5} of coal sample 71.
and a control and computation unit (2) that receives the measurement signals W_{coal5} and H_{coal5}, computes the density

$${ρ}_{coal measured 5} = \frac{{W}_{coal 5}}{{H}_{coal 5} * {S}_{5}} ,$$

where S₅ is the bottom area of the measured coal sample.

(ρ_{coal measured5} corresponds to the described signal U and ρ_{coal}). Based on the ρ_{coal measured5}, identifies the corresponding coal quality parameters through the analysis software application.

### Embodiment 6—Construction of a Laser Scale Density Measuring Device 1f

A laser scale is used to measure the weight W_{coal6} of coal or coal sample on the conveyor belt, while an angular-displacement thickness measurement device is used to measure the thickness H_{coal6} of coal or coal sample.

FIG. 8 illustrates a schematic diagram of an online coal quality parameter analyzer constructed using the laser scale density measurement device. The laser scale coal quality parameter analyzer 3f comprises:
a shaping hopper (31) for shaping the coal or coal sample (71) into a stream of uniform width and thickness.
an angular-displacement thickness measurement device (32) for measuring the thickness H_{coal6} of the coal sample 71 on the belt.
a laser scale (33) consisting of a laser generator (331) and laser detector (332) for measuring the weight W_{coal6} of coal or samples on the belt.
and a control and computation unit (2) that receives the measurement signals W_{coal6} and H_{coal6}, computes the density

$${ρ}_{coal measured 6} = \frac{{w}_{coal 6}}{{H}_{coal 6} * {S}_{6}} ,$$

where S₆ is the bottom area of the measured coal sample.

(ρ_{coal measured6} corresponds to the described signal U and ρ_{coal}). Based on the ρ_{coal measured6}, identifies the corresponding coal quality parameters through the analysis software application.

FIG. 2B illustrates schematic diagrams of analyzers constructed using the different types of density measurement devices described above.

The steps for construct the big data coal quality parameter databases are detailed in the “Detailed Description”—“Technical Concept”—Construct Coal Quality Parameter Databases” section.

### Advantages of the Big Data-Enabled Coal Quality Parameters Analysis Method

1. The

$${ρ}_{coal} ( \frac{1 g}{{v}_{g}} ) ∼ {( A , M , V , FC , Q , S )}_{i} and U ∼ {ρ}_{coal} ( \frac{1 g}{{v}_{g}} )$$

databases constructed based on large amounts of data (A, M, V, FC, Q S)ᵢ, compared with building a mathematical measurement model in the prior techniques, are more comprehensive, more accurate, and highly consistent, reflecting the relationship between two variables in a one-to-one relationship, greatly improving the correlation between the two variables and achieving higher measurement accuracy.

2. The coal quality parameter intelligent analysis software operates without constructing mathematical models. It remains unaffected by variations in coal type or quality, as well as by interdependencies among multiple parameters. This approach overcomes the limitations of prior measurement methods and delivers enhanced accuracy in coal quality parameters assessment.

3. The density-based coal quality parameter analysis method utilizes a single variable, U, to enable rapid and accurate identification of six critical coal quality parameters: ash (A), moisture (M), volatile matter (V), fixed carbon (FC), calorific value (Q), and sulfur content (S), by leveraging big data databases. This approach represents the core technical innovation of the present invention. Compared to conventional techniques, it offers significant improvements in analytical efficiency and precision.

It will be understood by those skilled in the art that various modifications, substitutions, and alterations may be made to the disclosed invention without departing from its spirit or scope. Such variations and equivalent implementations are intended to be covered by the claims appended hereto and fall within the scope of protection sought under United States patent law.

What is claimed is:

1. A big data-enabled density-based coal quality parameter analysis method, the method comprising:
using a coal quality parameter measurement device to measure a coal quality parameter variation signal U of coal or coal sample, the signal U serving as a variable input for analysis; and
identifying corresponding coal quality parameters, including at least one of ash (A), moisture (M), volatile matter (V), fixed carbon (FC), calorific value (Q), or sulfur (S), corresponding to a value of the signal U via a big data-driven coal quality parameter databases,
wherein the identifying coal quality parameters corresponding to U via the big data-driven coal quality parameter databases comprises:
Step 1:
using the coal quality parameter variation signal U, measured by the coal quality parameter measurement device, as a variable; and
utilizing big data technology to construct one or more databases based on a relationship
$${ρ}_{coal} ( \frac{1 g}{{v}_{g}} ) = {ρ}_{A} * A + {ρ}_{M} * M + {ρ}_{V} * V + {ρ}_{FC} * FC ,$$
using large datasets of measured A, M, V, FC, Q, S values by applying national standards methods across countries, represented as (A, M, V, FC, Q, S)ᵢ, i=1, 2, 3 . . . n, the one or more databases being designed to store coal quality parameters and corresponding
$${ρ}_{coal} ( \frac{1 g}{{v}_{g}} )$$
and coal quality parameter variation signal U measurement data, and being adaptable to various coal types and the intelligent analysis software application analytical requirements,
where:
$${ρ}_{c o a l} ( \frac{lg}{Vg} )$$
denotes coal or coal sample's density;
A, M, V, and FC denote coal quality parameters, including ash, moisture, volatile matter, fixed carbon, respectively;
Q denotes calorific value of coal; S denotes sulfur content in coal; and
ρ_{A}, ρ_{M}, ρ_{V}, and ρ_{FC} denote ash density, moisture density, volatile matter density, and fixed carbon density of coal, respectively;
Step 2:
employing identification technology using the variation signal U of the coal or coal sample measured by the coal quality parameter measurement devices, and statically measured coal or coal sample's quality parameters A, M, V, FC, combined with the one or more databases storing coal quality parameters, to develop an intelligent analysis software application; and
Step 3:
constructing a coal quality parameter analyzer comprising a measurement device and an integrated control and computation unit, the coal quality parameter measurement device capturing the coal quality parameter variation signal U, transmitting the variation signal U to the control and computation unit, which processes U using the intelligent analysis software application, and through recognition and databases matching, identifies the corresponding coal quality parameters A, M, V, FC, Q, S.

2. The big data-enabled density-based coal quality parameter analysis method according to claim 1, wherein the constructing the one or more databases using big data technologies, designed to store coal quality parameters and measurement data adaptable to various coal types and analytical requirements comprises:
constructing a
$${ρ}_{coal} ( \frac{1 g}{{v}_{g}} ) ∼ {( A , M , V , FC , Q , S )}_{i}$$
database;
constructing a
$$U ∼ {ρ}_{coal} ( \frac{1 g}{{v}_{g}} )$$
database;
constructing a U~(A, M, V, FC, Q S)ᵢ database;
constructing a
$${ρ}_{coal} ( \frac{1 g}{{v}_{g}} ) ∼ {( A , M , V , FC , Q , S )}_{i}$$
parameter measurement database;
constructing a
$$U ∼ {ρ}_{coal} ( \frac{1 g}{{v}_{g}} )$$
parameter measurement database;
constructing a U~(A, M, V, FC, Q S)ᵢ parameter measurement database; and
constructing
$$A ∼ {ρ}_{coal} ( \frac{1 g}{{v}_{g}} ) , M ∼ {ρ}_{coal} ( \frac{1 g}{{v}_{g}} ) , V ∼ {ρ}_{coal} ( \frac{1 g}{{v}_{g}} ) , FC ∼ {ρ}_{coal} ( \frac{1 g}{{v}_{g}} ) ,$$
$$Q ∼ {ρ}_{coal} ( \frac{1 g}{{v}_{g}} ) , and S ∼ {ρ}_{coal} ( \frac{1 g}{{v}_{g}} )$$
parameter measurement databases.

3. The big data-enabled density-based coal quality parameter analysis method according to claim 1 wherein the developing the intelligent analysis software application comprises:
based on the coal quality parameter variation signal U, using the
$$U ∼ {ρ}_{coal} ( \frac{1 g}{{v}_{g}} )$$
database to develop software code to identify U, getting the
$${ρ}_{coal} ( \frac{1 g}{{v}_{g}} ) ;$$
and
based on the
$${ρ}_{coal} ( \frac{1 g}{{v}_{g}} ) ,$$
using the
$${ρ}_{coal} ( \frac{1 g}{{v}_{g}} ) ∼ {( A , M , V , FC , Q , S )}_{i}$$
database to develop the software code to identify
$${ρ}_{coal} ( \frac{1 g}{{v}_{g}} ) .$$

4. A coal quality parameter analyzer constructed based on the big data-enabled density-based coal quality parameter analysis method of claim 1, the coal quality parameter analyzer including:
the coal quality parameter measurement device configured to measure the coal quality parameters variation signal U; and
the control and computation unit, comprising an Instrument Control Computer (ICC) and a Data Analysis Computer (DAC), the ICC managing the measurement process and outputting the coal quality parameters variation signal U, the DAC receiving the signal U, using big data-driven coal quality parameter databases and employing the intelligent analysis software application to identify the coal quality parameters corresponding to U.
